(19) United States
(12) Patent Application Publication
(10) Pub. No.: US 2017/0312171 A1
(43) Pub. Date: Nov. 2, 2017

(54) METHODS AND DEFIBRILLATORS UTILIZING HIDDEN MARKOV MODELS TO ANALYZE ECG AND/OR IMPEDANCE SIGNALS

(71) Applicant: University of Washington, Seattle, WA (US)

(72) Inventors: Heemun Kwok, Seattle, WA (US); Jason Coult, Seattle, WA (US); Lawrence D. Sherman, Seattle, WA (US)

(73) Assignee: University of Washington, Seattle, WA (US)

(21) Appl. No.: 15/526,218

(22) PCT Filed: Nov. 20, 2015
(86) PCT No.: PCT/US15/61947

§ 371 Date: May 11, 2017

Related U.S. Application Data
(60) Provisional application No. 62/082,776, filed on Nov. 21, 2014.
(60) Provisional application No. 62/153,888, filed on Apr. 28, 2015.

(51) Int. Cl. A61H 31/00; A61B 5/0402; A61N 1/39; A61B 5/0205; A61B 5/00; A61B 5/04; A61B 5/053

(52) U.S. Cl. CPC A61H 31/005; A61B 5/04012; A61B 5/0402; A61N 1/3925; A61N 1/3993; A61N 1/3987; A61B 5/0205; A61H 31/007; A61B 5/7264; A61B 5/053; A61H 2201/5084; A61H 2230/045; A61H 2201/5061

(57) ABSTRACT

Examples described herein include defibrillators or other medical equipment that may employ hidden Markov models to classify cardiac rhythms in ECG signals. Hidden Markov models may additionally or instead be used to determine presence of a chest compression from the thoracic impedance signal. Classification of cardiac rhythms may be used to determine when to deliver a shock to a patient. Other applications are also described.

### CROSS-REFERENCE TO RELATED APPLICATION(S)

This application claims the benefit of the earlier filing dates of U.S. Provisional Applications 62/082,776, filed Nov. 21, 2014 and 62/153,888, filed Apr. 28, 2015. The entire contents of both provisional applications are hereby incorporated by reference in their entirety, for any purpose.

### TECHNICAL FIELD

Examples described herein generally relate to defibrillators and other medical equipment that may analyze ECG signals and classify heart rhythms. Examples may include use of hidden Markov models in analyzing ECG and/or impedance signals, classifying heart rhythms, detecting chest compressions, and/or instructing regarding shock or resuscitation therapies.

### BACKGROUND

During resuscitation following cardiac arrest and other critical illnesses and injuries, the cardiac rhythm can change dynamically between different rhythms, such as shockable rhythms (e.g., ventricular fibrillation), organized rhythms (rhythms with organized ventricular activity, such as sinus rhythm and atrial fibrillation), and asystole. Ventricular fibrillation and asystole do not have any discrete ECG waves (e.g. P waves or QRS complexes) or defined cardiac cycles. These changes in cardiac rhythm are important to identify both rapidly and accurately. Cardiac defibrillators are used to monitor the ECG continuously, but artifact from chest compressions, patient movement and other sources can obscure the cardiac rhythm. Therefore, there is a need for automated ECG signal processing methods which can classify the cardiac rhythm in the presence of artifact. Current signal processing methods are generally not accurate enough to be employed in the clinical setting for continuous ECG classification.

Previously described automatic rhythm classification methods analyze short ECG segments (e.g., five seconds) in isolation and assume a stable rhythm within that segment. Such assumptions and use of isolated segments may provide inaccurate results in some instances. Other methods can be used to classify organized rhythms, but not rhythms without defined ECG waves, such as ventricular fibrillation.

### SUMMARY

Examples of methods are described herein. An example method includes obtaining an electrocardiogram (ECG) signal, extracting features from the ECG signal, and applying a hidden Markov model to the features from the ECG signal to calculate a probability of a current frame having a particular state. The probability of the current frame having the particular state is based on probabilities previous frames of the ECG signal had certain states.

Some examples further include updating the probabilities at least one of the previous frames had the certain states based on the current frame of the ECG signal.

In some examples, each of the frames corresponds with ECG signal data collected over a time period at least two seconds.

Some examples further include detecting chest compression occurring during the current frame, and comparing the features with representative features based on the chest compression being detected.

In some examples, the representative features comprise a first set of features if the chest compression is detected and a second, different, set of features if the chest compression is not detected.

In some examples, detecting chest compression comprises receiving an impedance signal between electrodes used to provide the ECG signal, and some examples further include calculating a probability that chest compression is occurring by applying another hidden Markov model to the impedance signal.

Some examples further include providing an indication to shock a patient when a probability the current frame corresponds with a shockable rhythm meets or exceeds a threshold probability.

In some examples, applying the hidden Markov model comprises combining a sequence prior probability and a sequence likelihood to generate a sequence posterior probability.

Some examples further include accessing the sequence prior probability from electronic storage, wherein the sequence prior probability is based on a training set.

Some examples further include calculating the sequence likelihood using an emission distribution for each state of a sequence of frames.

Examples of defibrillators are described herein. An example defibrillator includes electrodes configured for application to a chest of a patient, memory configured to store statistical data relating to a hidden Markov model, hardware, software, firmware, or a combination thereof configured to receive an ECG signal from the electrodes and apply the hidden Markov model to the ECG signal and provide a probability the ECG is indicative of a shockable rhythm, and a display configured to provide an indication to shock the patient when the probability exceeds a threshold.

In some examples, the hardware, software, firmware, or combination thereof is configured to apply the hidden Markov model in part by comparing features extracted from the ECG signal to representative features. The representative features may be selected based on whether compressions are being performed on the patient.

In some examples, the hardware, software, firmware, or combination thereof is further configured to receiving an impedance signal from the electrodes and apply another hidden Markov model to the impedance signal and provide another probability the impedance signal is indicative of compressions being performed on the patient.

In some examples, the memory is further configured to store statistical data relating to a hidden Markov model.

In some examples, the statistical data is developed from a training set.

Examples of software are described that may include at least one non-transitory computer readable medium encoded with instructions, that, when executed, cause at least one processing unit to perform actions. Examples of actions include analyze a plurality of frames of ECG data, update respective probabilities for each of the plurality of frames of ECG data. The respective probabilities may indicate whether each frame of the ECG data reflects a particular cardiac rhythm classification. At least one of the respective probabilities may be updated based on ECG data from one of the plurality of frames occurring later in time.

In some examples, analyze a plurality of frames comprises using a hidden Markov model.

In some examples, each of the plurality of frames corresponds to a portion of ECG data representing at least two seconds of time.

In some examples, the respective probabilities are updated based on overall probabilities of the plurality of frames having a sequence of cardiac rhythm classifications.

In some examples, analyze a plurality of frames of ECG data comprises compare features from the frames of ECG data with representative features, the representative features for each frame selected based on whether chest compression was occurring during the frame.

### BRIEF DESCRIPTION OF THE DRAWINGS

FIG. 1 is a flowchart of a method arranged in accordance with examples described herein.

FIG. 2 is a schematic illustration of a relationship between hidden states and ECG signal features arranged in accordance with examples described herein.

FIG. 3 is a schematic illustration of transition probabilities between rhythm states in accordance with examples described herein.

FIG. 4 is a schematic illustration of a CPR scenario arranged in accordance with examples described herein.

FIG. 5 is a schematic block diagram of defibrillator arranged according to examples described herein.

### DETAILED DESCRIPTION

Certain details are set forth below to provide a sufficient understanding of embodiments of the invention. However, it will be clear to one skilled in the art that embodiments of the invention may be practiced without various of these particular details. In some instances, well-known circuits, control signals, timing protocols, and software operations have not been shown in detail in order to avoid unnecessarily obscuring the described embodiments of the invention.

Hidden Markov modeling and hidden Markov models generally refer to statistical methods used to analyze sequential data and perform integration. Hidden Markov models have been applied to the analysis of organized rhythms (e.g. sinus rhythm, atrial fibrillation and premature ventricular complexes) by modeling the sequence of ECG waves (e.g., P, QRS, and T waves) within a single cardiac cycle in order to characterize aspects about an individual heartbeat. These previous methods thus depend on observation of discrete ECG waves, which are typically obscured by chest compression artifact, and are therefore neither intended, nor feasible, for use during cardiac arrest resuscitation. Examples described herein utilize Hidden Markov models to classify rhythms without ECG waves or defined cardiac cycles, e.g. ventricular fibrillation and asystole, which are important causes of cardiac arrest. Alternately stated, examples described herein may operate independent of whether a waveform of a cardiac cycle is present in an input signal.

Methods described herein include examples of methods to classify the cardiac rhythm in a continuous manner from an electrocardiogram (ECG) signal by applying a class of models known as hidden Markov models. There are a variety of signal processing methods which classify ECG segments, but previously described “static” methods assume that a particular ECG segment has a stable (e.g. fixed) rhythm and analyzes each segment in isolation of others. However, during resuscitation, the cardiac rhythm can change, and these changes may be important to identify. Examples described herein utilize hidden Markov models to classify the cardiac rhythm dynamically. The use of hidden Markov models described herein may be employed in conjunction with any signal processing technique that extracts features from the ECG signal. Unlike static methods, the use of hidden Markov models integrates information from multiple signal frames within a continuous ECG segment to classify the rhythm sequence as a whole with dynamic rhythm transitions possible.

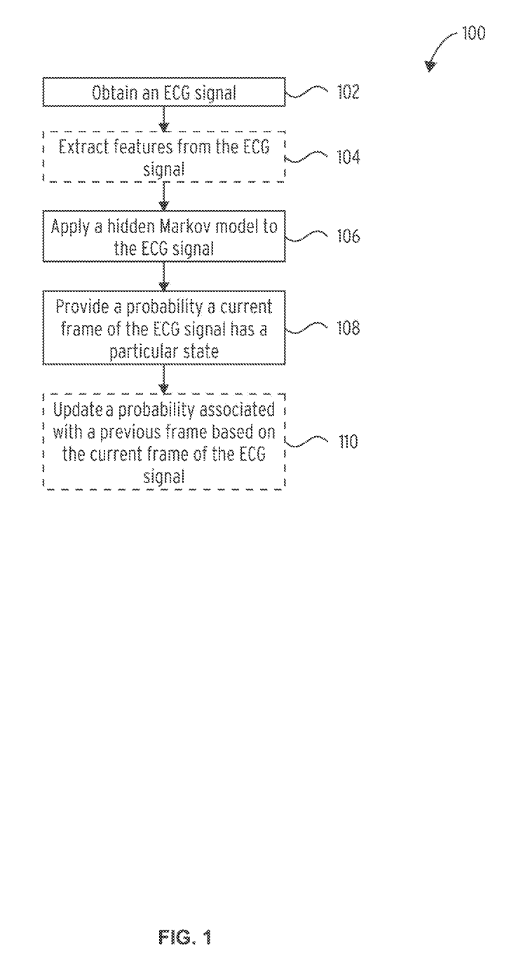

FIG. 1 is a flowchart of a method arranged in accordance with examples described herein. The method 100 includes obtain an ECG signal at block 102, optionally extract features from the ECG signal at block 104, apply a hidden Markov model to the ECG signal at block 106, provide a probability a current frame of the ECG signal has a particular state at block 108, and update a probability associated with a previous frame based on the current from of the ECG signal at block 110.

The method 100 may be implemented in hardware, software, firmware, or combinations thereof. The method 100 may be performed generally by any computing device and may be incorporated, for example, in a defibrillator, computer, server, tablet, mobile device, wearable device, or appliance. Other medical equipment may in some examples be used to implement the method 100.

In block 102, an electrocardiogram (ECG) signal may be obtained. The ECG signal may be obtained directly from a patient (e.g. through electrodes applied to a patient's chest). In some examples, the ECG signal may be obtained by accessing a stored ECG signal in electronic memory (e.g. memory local to a computing device or remote storage). The ECG signal may be considered in frames of data. Frames of data generally represent groups of data corresponding to temporal intervals. For examples, frames may represent 1 second, 2 seconds, 3 seconds, 4 seconds, 5 seconds, or 10 seconds of data. Other durations of data may be used per frame in other examples. Note that frames described herein may include data having a duration longer than a single cardiac cycle and/or include at least a plurality of cardiac cycles. Generally, examples described herein are interested in rhythm classification occurring over longer than a single cardiac cycle or for rhythms with an undefined cardiac cycle (e.g. ventricular fibrillation or asystole)

In block 104, features may be optionally extracted from the ECG signal. Generally, extracting features may include identification of relevant signatures in the ECG signal that may aid in classification of the signal as associated with a certain rhythm. Examples of features include, but are not limited to, amplitudes, peaks, frequency powers, frequency distributions, entropies, and durations. In this manner, the entire ECG signal data may not be needed to conduct classification of a rhythm. Instead, features of the ECG signal may be extracted and used for classification.

Generally, the classification may include comparing the extracted features with features representative of certain cardiac rhythm states. Cardiac rhythms of interest include, but are not limited to, asystole, organized rhythm, and shockable rhythms. Each rhythm may have representative features indicative of the rhythm which may be stored and accessed during example methods described herein to compare the representative features with features in the obtained ECG signal to assist in classifying the rhythm.

The representative features used to compare with the obtained ECG signal may vary based on whether chest compression is occurring during the frame of data being compared. Accordingly, in some example methods described herein, a determination may be made whether chest compression is occurring. In some examples, an impedance signal provided between two electrodes used to obtain the ECG signal (or other electrodes in some examples) is analyzed to determine whether chest compression is occurring. The determination of whether chest compression is occurring based on the impedance signal may also utilize hidden Markov models in some examples.

In block 106, a hidden Markov model is applied to the ECG signal. This may be implemented, for example, by applying a hidden Markov model to features extracted from the ECG signal. The cardiac rhythm (e.g. asystole, organized rhythm, shockable rhythm) may be modeled as a sequence of “hidden” rhythm states, with each state corresponding to a different frame of ECG data. The hidden states are unknown, but can be learned about through the observed ECG signal features.

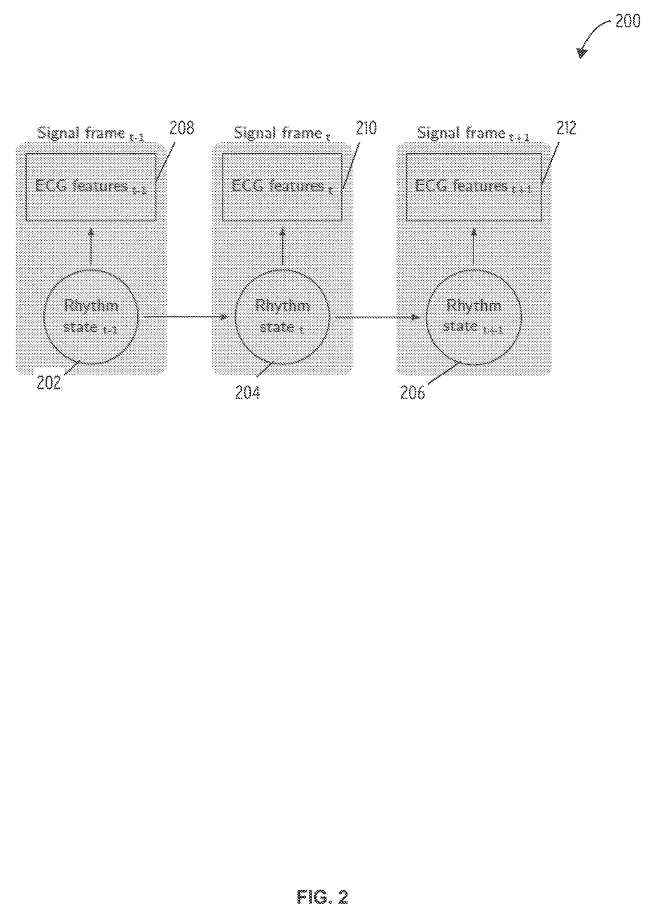

FIG. 2 is a schematic illustration of a relationship between hidden states and ECG signal features arranged in accordance with examples described herein. FIG. 2 illustrates three frames of ECG data: frame t−1, frame t, and frame t+1. The rhythm states associated with each frame are shown as state 202 for frame t−1, state 204 for frame t, and state 206 for frame t+1. Each of the states 202, 204, and 206 may not be known but may be analyzed using the associated ECG features. The ECG features 208 are shown associated with the frame t−1 and state 202. The ECG features 210 are shown associated with the frame t and state 204. The ECG features 212 are shown associated with the frame t+1 and state 206. The states (e.g. state 202, 204, and 206) will generally correspond with one of the possible cardiac rhythms (e.g. asystole, organized rhythm, shockable rhythm). Each state generally has a unique emission distribution of observed signal features. The emission distributions may adopt a variety of possible mathematical forms (e.g., a multivariate normal distribution). In this manner, a comparison of ECG signal feature (e.g. features 208, 210, and/or 212) with representative features for each of the possible states may yield a prediction (e.g. a probability) that the ECG signal frame corresponds with that state.

Generally, the emission distribution parameters (e.g. representative features) may be obtained from a training set. For example, a population of ECG signals from known rhythms may be analyzed and the distribution parameters associated with each rhythm may be derived and stored as statistical data. The statistical data may be used in later classifying obtained ECG signals. Recall also the representative features used for comparison may change depending on whether chest compression is being performed during the frame.

A hidden Markov model generally also models probabilistically how the rhythm states change dynamically (e.g. over time). This model may be provided in some examples using the assumption that the state sequence is a first-order Markov chain. Transitions from a rhythm state at a given time to all possible states at the next discrete time are governed by transition probabilities.

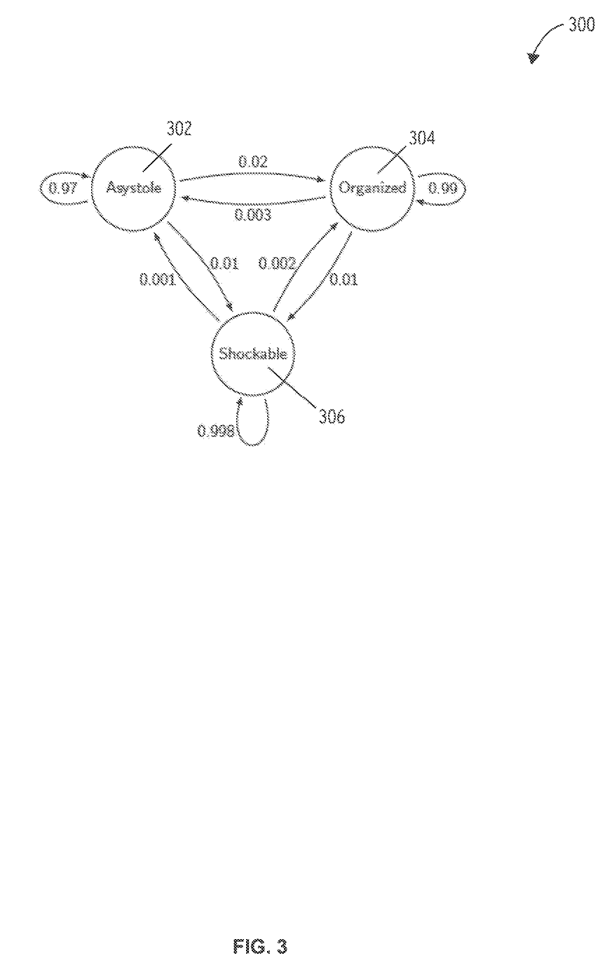

FIG. 3 is a schematic illustration of transition probabilities between rhythm states in accordance with examples described herein. FIG. 3 depicts a state diagram 300. The state diagram 300 includes an asystole state 302, an organized rhythm state 304, and a shockable rhythm state 306. The probabilities of transitions between states are shown with arrows in FIG. 3. In the example of FIG. 3, if a current frame is an asystole state 302, a probability the next frame will also be an asystole state is 97 percent, the probability the next frame will be organized rhythm state 304 is 2 percent, and the probability the next frame will be shockable rhythm state 306 is 1 percent. If a current frame is organized rhythm state 304, the probability the next frame will be the organized rhythm state 304 is 99 percent, the probability the next frame will be the asystole state 302 is 0.3 percent, and the probability the next frame will be the shockable rhythm state 306 is 1 percent (note some rounding approximations in these state transitions for example purposes). If a current frame is shockable rhythm state 306, the probability the next frame will be shockable rhythm state 306 is 99.8 percent, the probability the next frame will be asystole state 302 is 0.1 percent and the probability the next frame will be organized rhythm state 304 is 0.2 percent.

In this manner, a hidden Markov model will provide a number of possible states (e.g. asystole, shockable rhythm, organized rhythm) and for each state, provide a probability that the next state will be each of the possible states. The state transition probabilities may be determined from a training set. For example, ECG signals from known rhythms may be analyzed as to state transitions and the overall state transition probabilities from the analyzed ECG signals from known rhythms may be stored and used for classification of later obtained ECG signals. The state transition probabilities may be stored as statistical data usable in classifying ECG signals.

Another component of a hidden Markov model is a distribution of initial states, which may also be referred to as the initial state distribution. The initial state is the state of the first frame of the observed ECG signal in some examples. The hidden Markov model may specify a probability that the initial state is each of the possible states—e.g. a probability the initial state is the asystole state, a probability the initial state is the organized rhythm state, and a probability the initial state is the shockable rhythm state. The distribution of initial states may also be determined empirically from a training set and stored as statistical data usable in classifying ECG signals.

Accordingly, hidden Markov models described herein may include state definitions which correlate hidden states with features from ECG signals (see, e.g., FIG. 2). Correlations between representative features and states may be stored as statistical data. Hidden Markov models described herein may additionally or instead include state transition probabilities describing a statistical probability of transition from one certain state to another (see, e.g. FIG. 3). State transition probabilities may be stored as statistical data. Hidden Markov models described herein may additionally or instead include a distribution of initial states. The distribution of initial states may be stored as statistical data. The statistical data may be used to dynamically classify obtained ECG signals.

Because a hidden Markov model is a full probabilistic model, Bayes' rule can be used to find the probability of any particular rhythm sequence S={St, t=1, . . . , T}, where St is the rhythm state at time t and T is the number of frames within the segment. The sequence prior probability P(S) is the probability of the sequence given the initial state distribution P(S0) and the transition probability matrix P(St|St−1). The sequence prior probability P(S) does not rely on any ECG data, and may be given as follows:

$$P ( S ) = P ( {S}_{0} ) {\prod }_{t = 1}^{T} P ( {S}_{t} \vert {S}_{t - 1} ) .$$

Accordingly, without any information from the obtained ECG signal, the probability of any possible sequence may be given by P(S) based on stored statistical data. The probability of the sequence P(S) may be calculated using the equation above, recognizing that the probability of the sequence is the probability of the initial state multiplied by the probabilities of each subsequent state transition for the sequence. In some examples, the sequence prior probabilities P(S) may be stored as statistical data usable in classifying obtained ECG signals.

The sequence likelihood P(X|S) is the sampling probability of the ECG data, which includes the sequence of vectors X={Xt, t=1, . . . , T}, where Xt is the vector of ECG features at time t. The sequence likelihood is calculated using the emission distribution P(Xt|St) for each state St of the sequence as follows:

P(X|S)=Π_{2*1}^{p}P(X₂|S₂),

While the vector X has been described as a vector of ECG features, in some examples, the ECG data may include additional information. Information which may also be included in the vector may include, but is not limited to, accelerometer data, impedance signal data (e.g. impedance between electrodes used to obtain the ECG signal), GPS data, or combinations thereof.

For example, the sequence likelihood may represent the likelihood of the features in the obtained ECG signal given a possible sequence. The sequence likelihood is given by a product of the probabilities the extracted features in each frame correspond to the state allotted that frame in the sequence being reviewed.

Accordingly, two metrics may be available for each sequence of frames. Looking at all possible sequences for that sequence of frames, each sequence may have a sequence prior probability based only on statistical data without use of information (e.g. features) from the ECG signal itself. Each sequence may also have a sequence likelihood calculated using only information (e.g. features) from the ECG signal and without use of information relating to the statistical likelihood of the sequence.

Bayes' rule combines the sequence prior probability and sequence likelihood to calculate the sequence posterior probability P(S|X), which is the probability of the sequence given the obtained ECG signal, as follows:

$$P ( S \vert X ) = \frac{P ( S , X )}{P ( X )} = \frac{P ( X \vert S ) P ( S )}{P ( X )}$$

P(X) is the sum of the joint probabilities P(S,X) over all possible sequences, as follows:

P(X)=Σ_{2Hp}P(X|S)P(S),

The method sequence classification is the sequence with the highest posterior probability. For example, methods described herein may calculate a sequence posterior probability for each possible sequence for a given plurality of frames of obtained ECG signal. Example methods may select the sequence having the highest sequence posterior probability as the correct sequence and may classify the frames of the obtained ECG signal as belonging to the corresponding states specified by the correct sequence. Because the number of possible sequences increases exponentially with the number of frames within an ECG segment, the Viterbi algorithm (a dynamic programming algorithm) may be used to efficiently search for it.

For example, consider a 24-second segment of ventricular fibrillation during CPR. The segment may include six signal frames, each lasting four seconds. With three possible rhythm states for each frame, there are 3⁶ or 729 possible rhythm sequences. The true sequence, {shockable-shockable-shockable-shockable-shockable-shockable}, is one possible sequence, and based on the transition probabilities for this example, its prior probability is 0.33. The sequence likelihood is calculated from Xt and St for each of the six frames and the emission distribution, and, from Bayes' rule, the posterior probability of this sequence is determined to be 0.98.

It is to be understood that although specific hidden Markov models and parameters are described herein, generally any hidden Markov model may be used. Application of a hidden Markov model generally allows for classification of a given frame of ECG data to be influenced by the classification of all frames of the ECG data, and in this manner, may improve classification accuracy. For example, a hidden Markov model generally may assume a geometric distribution for the sojourn time. Hidden Markov models described herein may include a hidden semi-Markov model where the transition probabilities and sojourn time are related.

In examples described herein, the hidden states of hidden Markov models may be defined by cardiac rhythm. In some examples, other definitions of the hidden states may be used. For example, the hidden states could be defined by cardiac rhythm and the presence or absence of chest compressions; under this classification scheme, asystole in the presence of chest compressions and asystole in the absence of chest compressions would be different states, for example. Moreover, while the cardiac rhythm was classified as asystole, an organized rhythm or a shockable rhythm in the above example, other cardiac rhythm classifications may be used in other examples. While analysis of ECG signals has been described for classification, example methods described herein may be applicable to other physiologic signal data and in settings other than critical illness and resuscitation.

A hidden Markov model may be used to accurately identify the most likely sequence for a plurality of frames of ECG data. For some therapeutic decisions, however, the primary interest will be the state of a particular frame, most commonly the current frame, and not the sequence as a whole. Referring back to FIG. 2, in block 108, a hidden Markov model may be applied to the features from the ECG signal to calculate the forward probability of a current frame having a particular state, and the probability of the current frame having the particular state is based on features from the current and previous frames of the ECG signal.

In this manner, a frame may be classified as a certain rhythm when the forward probability indicates the frame be so classified. Methods described herein may further act on classifications of a current frame. For example, when a frame is classified as a shockable rhythm (e.g. when a probability the current frame corresponds with a shockable rhythm meets or exceeds a threshold probability), an indication may be provided to shock a patient (e.g. using a visual display and/or audible alert).

In block 110, a probability associated with a previous frame may be updated based on temporally later frames of the ECG signal. Specifically, the smoothed probability of a particular frame may be calculated with the hidden Markov model using features from the frame of interest, the preceding frames, and the succeeding frames of the ECG signal. The classification of a frame may be based upon these smoothed probabilities, and the updated classification may or may not correspond with the manner in which the frame was previously classified. Similarly, on classifying a sequence of frames as corresponding with a certain sequence, the entire sequence may be updated in accordance with that selection of sequence. In this manner, classified states of earlier frames may change based on newly analyzed, temporally later, frames in an ECG signal.

Examples described herein may include medical equipment, such as defibrillators, that may analyze ECG signals to classify rhythms and may provide indications regarding how to act on those classifications—e.g. when to shock a patient.

Recall with respect of FIG. 2 that application of a hidden Markov model to an ECG signal may vary in accordance with whether chest compressions are being performed. For example, performance of chest compressions during a frame may change the reference features to which the ECG features are compared in evaluating sequence likelihood. Alternatively or in addition, hidden Markov models may have different states for each cardiac rhythm (or certain cardiac rhythms) with and without chest compression.

The presence of chest compressions may be determined based on analysis of an impedance signal between two electrodes coupled to a chest of a patient. In some examples, the same electrodes used to provide the ECG signal may provide the impedance signal. Hidden Markov models may be used to classify impedance signals as chest compression/no chest compression.

Chest compressions are a primary component of cardiopulmonary resuscitation (CPR), and the quality of their delivery affects survival following sudden cardiac arrest. Measurement of chest compression quality, e.g. rate and length of pauses, may allow for improving the delivery of CPR. Force sensors accurately measure chest compression metrics, but cost and complexity have limited their adoption. In contrast, during cardiac arrest resuscitation, cardiac defibrillators with monitor pads attached to the torso are universally employed. Because the electrical impedance between pads varies with chest compressions, it can be used to derive chest compression metrics. However, the relationship between impedance and force varies across patients and providers, and other sources of chest wall movement can generate artifact in the impedance signal. Therefore, there is a need for signal processing methods to accurately derive chest compression metrics from the impedance signal.

Other methods in existence claim to provide various chest compression metrics based on an electrical impedance signal. However, previous methods have not been based on hidden Markov models, which is a class of statistical models used for sequential data and therefore allows for the integration of continuous information during the course of resuscitation.

Example methods described herein calculate various chest compression quality metrics from the impedance signal. Example methods may employ hidden Markov models, modeling the impedance signal using a sequence of “hidden states” at discrete times. The “hidden” states and interval between discrete observations may differ among implemented hidden Markov models, which may allow for the derivation of different chest compression metrics.

Some example hidden Markov models may be provided to detect whether or not chest compressions are being performed during a given frame or other interval of impedance signal data. Detection of whether chest compressions are being performed would allow for the calculation of chest compression fraction and detection of pauses in chest compressions. Moreover, the detection of chest compression may affect the hidden Markov models and/or the features used to implement hidden Markov models for classifying cardiac rhythms in ECG signals. The possible hidden states in hidden Markov models used to classify chest compression/no chest compression may be (1) chest compressions present and (2) chest compressions absent. The states may be defined at two second intervals. In some examples, the states may be defined at other intervals, including 1, 2, 3, 4, 5, 6, 7, 8, 9, or 10 second intervals. In some examples, the intervals are equal to the frame duration described above with reference to classifying ECG signals. The model may account for temporal dependence by assuming that the sequence of hidden states forms a homogeneous first-order Markov chain. Within each two-second interval, impedance features may be extracted from the impedance signal. Conditionally on the hidden states, emission distributions may be defined of these impedance features using training data in which the true states have been identified using a force signal, for example. Transition probabilities between possible states may be estimated using the available training data. The transition probabilities may be constant or dependent upon factors such as the similarity of the impedance features at adjacent times.

For any test impedance signal of arbitrary length, the sequence of states with the highest posterior probability may be identified using a dynamic programming algorithm known as the Viterbi algorithm. The Viterbi sequence defines the intervals of chest compressions and the intervals of absent chest compressions. For any particular frame, the forward and smoothed probabilities of each state (chest compressions present or chest compressions absent) may be derived from the hidden Markov model, and these probabilities may be used to provide feedback to rescuers.

Some example hidden Markov models may be provided to identify individual chest compressions as well as the compression and relaxation phases within each compression, given that chest compressions are being performed. Chest compression rate and duty cycle may be derived from this information. The possible hidden states are “compression phase,” “relaxation phase” and “neutral” and are defined at 0.05 second intervals. Other intervals may be used such as 0.1, 0.15, 0.2, or other intervals. Impedance features at each discrete time may be obtained. Maximum likelihood estimates of the hidden Markov model parameters (e.g. initial distribution of states, emission distributions, and transition probabilities) may be estimated by applying the Expectation-Maximization algorithm to training data. A hidden Markov model may implicitly assume a geometric distribution for the sojourn time in each state, but in this method, a different distribution for the sojourn time (e.g., Poisson distribution) may be modeled explicitly.

For any test impedance signal during CPR, the Viterbi algorithm may be used to find the sequence of states with the highest posterior probability. The Viterbi sequence may define the compression phase and relaxation phase of each individual chest compression.

Example methods described herein may be applied to an impedance signal in real-time, e.g., as the signal is collected, in order to guide clinical care. Chest compression quality metrics could be communicated to the provider using a variety of formats, audio prompts or visual displays.

Methods may be applied to an impedance signal in a retrospective or post-hoc manner in order to review the events of a resuscitation. This type of review could occur contemporaneously during a resuscitation, such as during the transfer of care from one provider to another. Case review with the impedance signal annotated using this method would also be useful at a later time for quality improvement, research, or educational purposes.

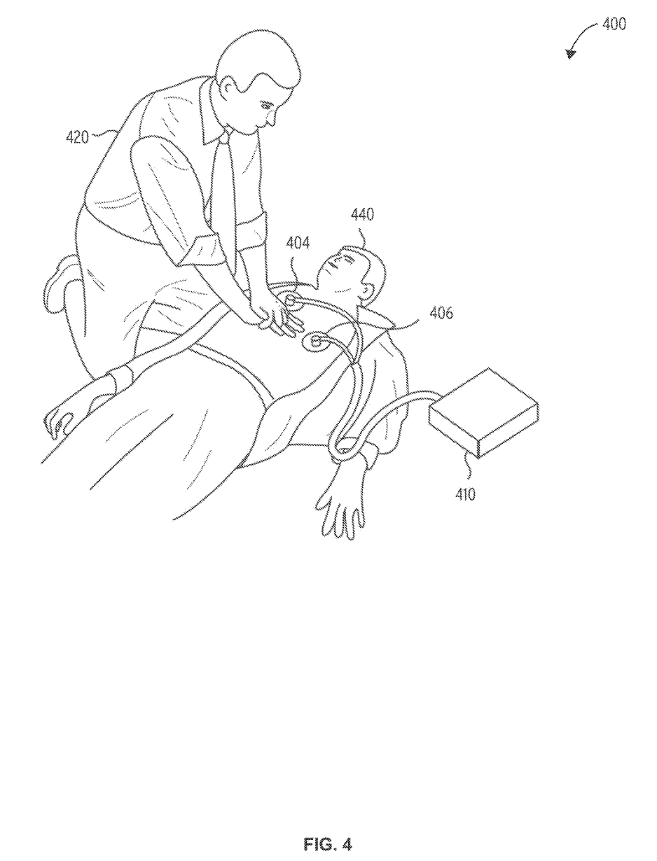

FIG. 4 is a schematic illustration of a CPR scenario arranged in accordance with examples described herein. FIG. 4 illustrates a responder 420 performing CPR on a patient 440 that is connected to an automated external defibrillator (AED) 410. In this scenario, the patient 440 may be exhibiting signs of cardiac arrest. The responder 420 may be a person that is trained in proper CPR techniques. In this example, the patient 440 may have two electrodes 404 and 406 applied to his/her chest. The two electrodes 404 and 406 may be attached to the skin of the patient 440 at conventional locations, such as one electrode 404 applied under the right collar bone and the other electrode 406 applied to left lower chest. The two electrodes 404 and 406 may be coupled to the AED 410 via a cable.

The AED 410 may obtain an ECG signal from the patient 440 via the two electrodes 404 and 406, including while the responder 420 is performing CPR. The AED 410 may analyze the ECG signal to classify the ECG rhythm of the patient 440 as shockable or non-shockable (or asystole in some examples). Other classifications may be used in other examples. Responsive to the classification of a shockable rhythm, the AED 410 may apply high-voltage (e.g. 1,200-1.800 volts) shocks, and/or may provide an audio and/or visual indication that shocks are recommended. While the AED is connected to the two electrodes 404 and 406 to detect the ECG signal, the responder 420 may perform CPR by applying downward forces or compressions to the sternum of the patient 440. In some instances, CPR may also include the responder 420 blowing air into the mouth or nose of the patient 440 by mouth-to-mouth or mouth-to-nose breathing. Analysis of the ECG signal may be dependent on whether chest compressions are being administered to the patient 440. That is, analysis of the ECG signal may be different when chest compressions are being administered than when no chest compressions are being administered. Thus, the AED 410 may further analyze an impedance signal between the electrodes 404 and 406 over time to provide a chest compression/no chest compression classification associated with the patient 440. In some examples, the AED 410 may prompt the responder 420 to stop chest compressions to allow for a shock to be administered to the patient 440.

The AED 410 may include hardware, software, firmware, or combinations thereof which are configured to classify ECG rhythms using hidden Markov models in accordance with examples described herein. For example, the hardware, software, firmware, or combinations thereof may receive an ECG signal from the electrodes and apply the hidden Markov model to the ECG signal and provide a probability the ECG is indicative of a shockable rhythm. Generally, the hardware, software, firmware, or combinations thereof may be configured to implement the methods described herein with reference to FIGS. 1-3. Additionally or instead, the AED 410 may include hardware, software, firmware, or combinations thereof which are configured to determine whether chest compressions are being administered to the patient 440 based on analysis of an impedance signal between the electrodes 404 and 406 over time. This information may be used to assist in analysis of the ECG signal, or to provide reminders to the responder 420 to administer CPR. In analyzing an ECG signal, chest compressions during CPR may introduce artifacts into the ECG signal, which may mask or obscure the underlying ECG rhythm, making classification of an ECG rhythm of the patient 420 difficult with others methods in existence, and thus the AED 410 may apply signal processing techniques to the impedance signal to determine whether chest compressions are being administered. The analysis may include utilizing hidden Markov models.

The hardware, software, firmware, or combinations thereof may be implemented, for example, using one or more processing unit(s) (e.g. processor(s)) and one or more computer readable mediums (e.g. memory) encoded with instructions, which when executed, cause the at least one processing unit(s) to perform the described actions. In some examples, the hardware, software, firmware, or combinations thereof may be implemented using custom ASIC or other circuitry configured to perform the described functions. In some examples, the hardware, software, firmware, or combinations thereof may be implemented using firmware configured to perform the described functions.

The methods disclosed herein may be implemented using hardware, software, firmware, or combinations thereof. For example, a field-programmable gate array (FPGA) device, an application-specific integrated circuit (ASIC), a processing unit such as a central processing unit (CPU), a digital signal processor (DSP), a controller, another hardware device, a firmware device, or any combination thereof may be used. As an example, the methods may be implemented by a computing system using, for example, one or more processing units that may execute instructions for performing the method that may be encoded on a computer readable medium. The processing units may be implemented using, e.g. processors or other circuitry capable of processing (e.g. one or more controllers or other circuitry). The computer readable medium may be transitory or non-transitory and may be implemented, for example, using any suitable electronic memory, including but not limited to, system memory, flash memory, solid state drives, hard disk drives, etc. One or more processing units and computer readable mediums encoding executable instructions may be used to implement all or portions of ECG classification systems, defibrillators, and/or ECG classification systems described herein.

In some embodiments, the hardware, software, firmware, or combinations thereof may implement other or different decision making methodologies. While the above describes a determination of whether chest compressions are being administered in an AED 410, the determination may be performed in other devices, such as an implantable defibrillator or an ECG monitor in a hospital setting that constantly or periodically monitors ECG signals via electrodes for evaluations over time or during a medical event.

While AED 410 is described as an automatic external defibrillator, which is generally designed for small physical size, light weight, and relatively simple user interface capable of being operated by personnel without high training levels, in other embodiments, the AED 410 may additionally or alternatively include other defibrillators, such as a manual defibrillator, an implantable defibrillator, a paramedic defibrillator, and/or a clinical defibrillator. Generally, paramedic or clinical defibrillators may be carried by an emergency medical service (EMS) responder, and tend to be larger, heavier, and have a more complex user interface capable of supporting a larger number of manual monitoring and analysis functions.

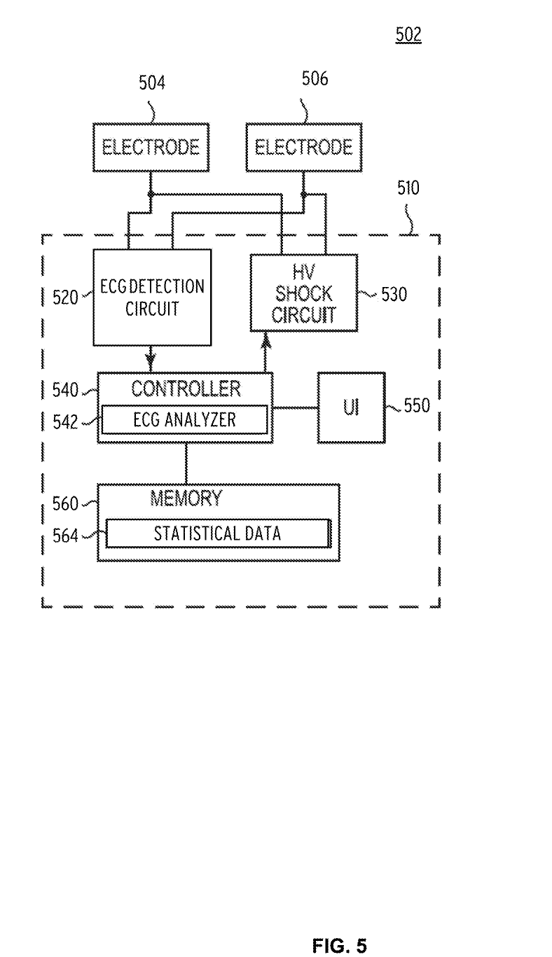

FIG. 5 is a schematic block diagram of defibrillator arranged according to examples described herein. The defibrillator system 502 may include a pair of electrodes 504 and 506 coupled to a defibrillator 510. The defibrillator 510 may be used to implement the AED 410 of FIG. 4.

The defibrillator 510 may include an ECG detection circuit 520 coupled to the pair of electrodes 504 and 506. The pair of electrodes 504 and 506 may be connected across the chest of a patient, such as the patient 440 of FIG. 4. The ECG detection circuit 420 may detect an ECG signal using the pair of electrodes 504 and 506. The ECG detection circuit 520 may provide the ECG signal to the controller 540. Additionally or instead, the defibrillator 510 may include an impedance detection circuit (which may be implemented also using the ECG detection circuit 520 an/or may be a separate detection circuit). The impedance detection circuit may detect an impedance between the electrode 504 and 506. The impedance detection circuit may provide the impedance to the controller 540.

The controller 540 may include an ECG analyzer 542 which may be implemented using hardware, software, firmware, or combinations thereof. The ECG analyzer 542 may utilize hidden Markov models to classify ECG signals as described herein. For example, the ECG analyzer may be used to implement the methods described with reference to FIGS. 1-3. The ECG analyzer 542 may additionally or instead determine whether chest compressions are being administered based on the impedance between the electrodes. The ECG analyzer 542 may provide a chest compression/no chest compression classification of the impedance signal to the controller 540. The controller 540 may analyze the ECG signal using a process that may be selected based the chest compression/no chest compression classification. The controller 540 may classify the ECG rhythm of the ECG signal of the patient as shockable, non-shockable, and/or asystole. If a shockable rhythm is detected (e.g., in combination with determination of a treatment regimen that indicates immediate defibrillation shock), the controller 540 may send a signal to high voltage (HV) shock circuit 530 to charge in preparation for delivering a shock. The AED 510 may include a user interface 550 that provides an indication to the controller 540 and/or to a user to administer the shock responsive to a user input. The user interface 550 may also provide an indication to the responder to begin or resume provision of CPR responsive to a signal from the controller 540 indicating that no CPR is detected by the ECG analyzer 542. The AED 510 may further include a memory 560 that is configured to store statistical data used for the classification by the ECG analyzer 542. For example, the statistical data may include statistical data relating to one or more hidden Markov models for classification of ECG signals and/or impedance signals. For example, the statistical data may include probabilities, initial state distributions, state transition probabilities, and/or other parameters relating to hidden Markov models, some or all of which may be derived from training sets.

In operation, the pair of electrodes 504 and 506 may be attached to a patient experiencing a medical event, such as cardiac arrest. The ECG detection circuit 520 and/or the impedance detection circuit may provide an ECG signal and/or impedance signal across the pair of electrodes 504 and 506 to continuously monitor the ECG signal and/or impedance between the pair of electrodes 504 and 506, including while the patient is receiving CPR or other medical care. The ECG detection circuit 520 may provide the ECG and/or impedance signal(s) to the controller 540. The ECG analyzer within the controller 540 may apply hidden Markov models to the ECG and/or impedance signals. The ECG analyzer 542 may further classify rhythms in the ECG signal (e.g. provide percentages indicative of states for frames of the ECG signal as described herein). The ECG analyzer 542 may provide an indication as to whether chest compressions are being administered to the controller 540. The controller 540 may analyze an ECG signal of the patient received via the pair of electrodes 504 and 506 to classify the ECG rhythm of the patient as shockable or non-shockable or asystole. An example of a shockable rhythm may include ventricular fibrillation. Examples of non-shockable rhythms may include asystole (e.g., flatline or state of no cardiac electrical activity), organized cardiac activity (e.g., normal sinus rhythm), or pulseless electrical activity (e.g., electrical signals indicate heart rhythm, but no pulse is produced). The process used in the analysis of the ECG signal may be based on whether chest compressions are being administered or not. If a shockable classification is determined, the controller 540 may send a command to the HV shock circuit 530 to begin charging. Responsive to an input at the user interface 550, the HV shock circuit 530 may release the high voltage to the electrodes 504 and 506 to administer a shock to a patient.

In some examples, systems may be provided which may analyze ECG signals that may be stored (e.g. previously observed ECG signals). This may be useful, for example, in a clinical or research setting where a researcher or practitioner desires to classify ECG signals previously obtained from a patient. Accordingly, computing systems may be provided (such as computers including, but not limited to, servers, laptops, desktops, mobile devices) that include at least one non-transitory computer readable medium encoded with instructions that, when executed, cause at least one processing unit to perform actions that implement ECG signal classification techniques described herein, such as those described with reference to FIGS. 1-3. For example, the instructions may include instructions to analyze a plurality of frames of ECG data and to calculate the forward and smooth probabilities of each frame of ECG data. The respective forward probabilities may indicate whether each frame of the ECG data reflects a particular cardiac rhythm state. The smooth probabilities include information from the plurality of frames occurring later in time and generally, at least one will differ from the forward probabilities. As described herein, the executable instructions may include executable instructions for evaluating a probability that the frames of ECG data correspond to a particular sequence of states. A sequence having a highest probability may be selected as a correct sequence, and each frame classified in accordance with the correct sequence.

The executable instructions may include instructions for analyzing a plurality of frames of ECG data using a hidden Markov model. Each of the plurality of frames analyzed may correspond to a portion of ECG data representing at least two seconds of time. In some examples, the respective probabilities are updated based on overall probabilities of the plurality of frames having a sequence of cardiac rhythm classifications.

In some examples, analyzing the plurality of frames of ECG data including comparing features from the frames of ECG data with representative features, the representative features for each frame selected based on whether chest compression was occurring during the frame.

From the foregoing it will be appreciated that, although specific embodiments of the invention have been described herein for purposes of illustration, various modifications may be made without deviating from the spirit and scope of the invention.

What is claimed is:

1. A method comprising:
obtaining an electrocardiogram (ECG) signal;
extracting features from the ECG signal;
applying a hidden Markov model to the features from the ECG signal to calculate a probability of a current frame having a particular state, wherein the probability of the current frame having the particular state is based on probabilities previous frames of the ECG signal had certain states.

2. The method of claim 1, further comprising updating the probabilities at least one of the previous frames had the certain states based on the current frame of the ECG signal.

3. The method of claim 1 wherein each of the frames corresponds with ECG signal data collected over a time period at least two seconds.

4. The method of claim 1 further comprising detecting chest compression occurring during the current frame, and comparing the features with representative features based on the chest compression being detected.

5. The method of claim 4, wherein the representative features comprise a first set of features if the chest compression is detected and a second, different, set of features if the chest compression is not detected.

6. The method of claim 4, wherein detecting chest compression comprises receiving an impedance signal between electrodes used to provide the ECG signal, and calculating a probability that chest compression is occurring by applying another hidden Markov model to the impedance signal.

7. The method of claim 1, further comprising providing an indication to shock a patient when a probability the current frame corresponds with a shockable rhythm meets or exceeds a threshold probability.

8. The method of claim 1 wherein applying the hidden Markov model comprises combining a sequence prior probability and a sequence likelihood to generate a sequence posterior probability.

9. The method of claim 8, further comprising accessing the sequence prior probability from electronic storage, wherein the sequence prior probability is based on a training set.

10. The method of claim 9, further comprising calculating the sequence likelihood using an emission distribution for each state of a sequence of frames.

11. A defibrillator comprising:
electrodes configured for application to a chest of a patient;
memory configured to store statistical data relating to a hidden Markov model;
hardware, software, firmware, or a combination thereof configured to receive an ECG signal from the electrodes and apply the hidden Markov model to the ECG signal and provide a probability the ECG is indicative of a shockable rhythm; and
a display configured to provide an indication to shock the patient when the probability exceeds a threshold.

12. The defibrillator of claim 1, wherein the hardware, software, firmware, or combination thereof is configured to apply the hidden Markov model in part by comparing features extracted from the ECG signal to representative features, wherein the representative features are selected based on whether compressions are being performed on the patient.

13. The defibrillator of claim 12, wherein the hardware, software, firmware, or combination thereof is further configured to receiving an impedance signal from the electrodes and apply another hidden Markov model to the impedance signal and provide another probability the impedance signal is indicative of compressions being performed on the patient.

14. The defibrillator of claim 13, wherein the memory is further configured to store statistical data relating to a hidden Markov model.

15. The defibrillator of claim 11, wherein the statistical data is developed from a training set.

16. At least one non-transitory computer readable medium encoded with instructions, that, when executed, cause at least one processing unit to perform actions comprising:
analyze a plurality of frames of ECG data;
update respective probabilities for each of the plurality of frames of ECG data, wherein the respective probabilities indicate whether each frame of the ECG data reflects a particular cardiac rhythm classification and
wherein at least one of the respective probabilities is updated based on ECG data from one of the plurality of frames occurring later in time.

17. The at least one non-transitory computer readable medium of claim 16, wherein said analyze a plurality of frames comprises using a hidden Markov model.

18. The at least one non-transitory computer readable medium of claim 16, wherein each of the plurality of frames corresponds to a portion of ECG data representing at least two seconds of time.

19. The at least one non-transitory computer readable medium of claim 16, wherein the respective probabilities are updated based on overall probabilities of the plurality of frames having a sequence of cardiac rhythm classifications.

20. The at least one non-transitory computer readable medium of claim 16, wherein said analyze a plurality of frames of ECG data comprises compare features from the frames of ECG data with representative features, the representative features for each frame selected based on whether chest compression was occurring during the frame.
